(19) United States
(12) Patent Application Publication
(10) Pub. No.: US 2021/0338820 A1
(43) Pub. Date: Nov. 4, 2021

(54) PHARMACEUTICAL COMPOSITION COMPRISING ANTIEMETIC COMPOUNDS AND POLYORTHOESTER

(71) Applicant: Heron Therapeutics, Inc., San Diego, CA (US)

(72) Inventors: Thomas B. Ottoboni, Belmont, CA (US); Lee Ann Lynn Girotti, San Bruno, CA (US)

(73) Assignee: Heron Therapeutics, Inc.

(21) Appl. No.: 17/121,567

(22) Filed: Dec. 14, 2020

Related U.S. Application Data
(63) Continuation of application No. 16/867,048, filed on May 5, 2020, now abandoned.
Application No. 16/867,048 is a continuation of application No. 15/588,034, filed on May 5, 2017, now abandoned.
Application No. 15/588,034 is a continuation of application No. 14/652,070, filed on Jun. 12, 2015, now abandoned.
(60) Provisional application No. 61/736,859, filed on Dec. 13, 2012.

(51) Int. Cl. A61K 47/34; A61K 31/439; A61K 31/5377; A61K 31/675; A61K 9/00; A61K 31/4178

(52) U.S. Cl. CPC A61K 47/34; A61K 31/439; A61K 31/5377; A61K 9/00; A61K 9/0024; A61K 31/4178; A61K 31/675

(57) ABSTRACT

The present disclosure provides for sustained release pharmaceutical formulations which can deliver both a 5-hydroxytryptamine 3 (5HT3) receptor antagonist and a neurokinin-1 (NK1) receptor antagonist to a subject in need thereof. Formulations described herein are suitable for subcutaneous administration. Also described are methods of treatment of various disorders, including chemotherapy-induced nausea and vomiting (CINV). The disclosed compositions and methods provide for less frequent dosing of the therapeutic agents, thereby increasing subject comfort and compliance.

### CROSS-REFERENCE TO RELATED APPLICATIONS

This application is a continuation of U.S. patent application Ser. No. 16/867,048, filed May 5, 2020, which is a continuation of U.S. patent application Ser. No. 15/588,034, filed May 5, 2017, which is a Continuation of U.S. patent application Ser. No. 14/652,070, filed Jun. 12, 2015, which is a U.S. National Stage of International Patent Application No. PCT/US2013/075153, filed Dec. 13, 2013, which claims the benefit of priority of U.S. Provisional Patent Application No. 61/736,859, filed Dec. 13, 2012, the content of each of which is incorporated herein by reference in its entirety.

### TECHNICAL FIELD

The current subject matter relates to pharmaceutical compositions for the controlled release of a 5-hydroxytryptamine 3 (5HT3) receptor antagonist and a neurokinin-1 (NK1) receptor antagonist and the use of such compositions in methods of treatment, including but not limited to treating chemotherapy induced nausea and vomiting (CINV).

### BACKGROUND

Nausea and vomiting which often follows chemotherapy is a severe and distressing side effect of many chemotherapeutics. Inadequate control of chemotherapy-induced nausea and vomiting (CINV) impairs patient function and activity and may interfere with treatment compliance. The antiemetic properties of 5-hydroxytryptamine 3 (5-HT3) receptor antagonists were discovered in the mid-1980s (e.g., Sanger et al., 1986, Br. J. Pharmacol., 88:497-499; Stables et al., 1987, Cancer Treat. Rev. 14:333-336). Four serotonin receptor antagonists, ondansetron, granisetron, dolasetron, and palonosetron, are currently available in the United States. Tropisetron, while not approved by the FDA, is available internationally. Despite the success of these therapeutics for the treatment of CINV, a significant number of patients receiving highly emetogenic chemotherapy such as cisplatin still suffer from CINV. One means of addressing this problem was the development of therapies with a different mechanism of action, for example, development of neurokinin-1 (NK-1) receptor antagonists. EMEND® (aprepitant) is an example of an NK-1 receptor antagonist which has proven to be effective in preventing emesis induced by chemotherapy (Navari et al., 1999, N Engl J Med, 340:190-195; Chawla et al., 2003, Cancer, 97:2290-2300).

With the intent of developing more effective treatments for CINV, studies were carried out to demonstrate the efficacy of combining the NK-1 receptor antagonist aprepitant with a 5-HT3 receptor antagonist (ondansetron) and a corticosteroid (dexamethasone). Results showed that addition of aprepitant to a standard treatment regimen of ondansetron and dexamethasone was generally well-tolerated and provided consistently superior protection against CINV in subjects receiving highly emetogenic cisplatin-based chemotherapy (Hesketh et al., 2003, J Clin Oncol. 21:4112-4119).

While such combination therapies have great promise in improving the quality of life of chemotherapy patients, currently approved therapies require multiple dosings on a daily basis. Accordingly, it would be beneficial to provide an antiemetic pharmaceutical formulation which is able to provide sustained release of the therapeutic agents, thereby reducing the number of administrations needed. Disclosed herein are compositions and methods which allow sustained release of antiemetic agents for the prevention or treatment of CINV and other disorders such as radiotherapy-induced nausea and vomiting and post-operative nausea and vomiting.

### BRIEF SUMMARY

The present disclosure is directed to pharmaceutical compositions and kits comprising a selective 5-hydroxytryptamine 3 (5-HT3) receptor antagonist as a first active agent and a neurokinin 1 (NK-1) receptor antagonist as a second active agent and corresponding methods of administration, treatment, and use. Both active agents can be in the same pharmaceutical dosage form or the two active agents can each be in a separate dosage form. The dosage form can be formulated for sustained or controlled release of the 5-HT3 receptor antagonist and/or the NK-1 receptor antagonist. The pharmaceutical compositions, kits and methods can be utilized in the treatment of nausea and vomiting, e.g., acute and delayed chemotherapy-induced nausea and vomiting (CINV).

In one aspect, there is provided a pharmaceutical composition for the sustained and controlled release of a therapeutically effective amount of a selective 5-HT3 receptor antagonist and a NK-1 receptor antagonist.

In one embodiment, the pharmaceutical composition comprises the 5-HT3 receptor antagonist and the NK-1 receptor antagonist in a single formulation for subcutaneous injection.

In yet embodiment, the pharmaceutical composition comprises the 5-HT3 receptor antagonist in a first pharmaceutical formulation and the NK-1 receptor antagonist in a second formulation. In a further embodiment, both the first and second formulations are formulated for subcutaneous injection. In one embodiment, the 5-HT3 receptor antagonist is granisetron or a pharmaceutically acceptable salt thereof or ondansetron or a pharmaceutically acceptable salt thereof.

In another embodiment, the NK-1 receptor antagonist is selected form the group consisting of aprepitant, fosaprepitant, rolapitant, netupitant, Ianepitant, vestipitant, orvepitant maleate, casopitant, ezlopitant, serlopitant and maropitant, or a pharmaceutically acceptable salt thereof.

In a particular embodiment, the pharmaceutical composition comprising the 5-HT3 receptor antagonist is a semi-solid composition.

In a particular embodiment, the pharmaceutical composition comprising the NK-1 receptor antagonist is a semi-solid composition.

In another particular embodiment, the pharmaceutical composition comprising the 5-HT3 and the NK-1 receptor antagonists are a semi-solid composition.

In one embodiment, the semi-solid composition comprises a polyorthoester, about 10-50 weight percent (wt %) of a polyorthoester compatible liquid excipient, and about 1-10 wt % of a 5-HT3 receptor antagonist. In another embodiment, the semi-solid composition comprises about 1-5 wt % or 2 wt %, 3 wt %, 4 wt %, or 5 wt % of the 5-HT3 receptor antagonist.

In one embodiment, the polyorthoester comprises subunits selected from

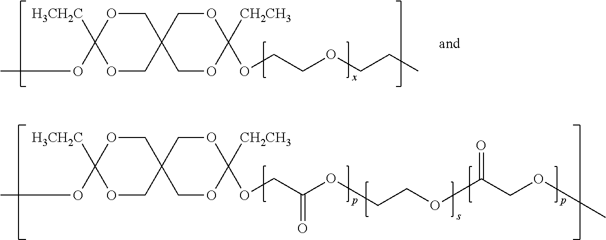

where

x is an integer from 1-4,

the total amount of p is an integer from 1-20,

s is an integer from 1-4,

the mole percentage of α-hydroxyacid containing subunits in the polyorthoester is from about 0.1 to about 25 mole percent, and the polyorthoester has a molecular weight in a range of about 1,000 Da to 10,000 Da. Exemplary molecular weights include 1,000 Da, 2,000 Da, 3,000 Da, 4,000 Da, 5,000 Da, 6,000 Da, 7,000 Da, 7,000 Da, 8,000 Da, 9,000 Da, 10,000 Da.

In yet a further embodiment, the semi-solid pharmaceutical composition comprises a polyorthoester, about 10-50 wt % of a polyorthoester-compatible liquid excipient, and about 1-5 wt % granisetron, wherein the polyorthoester comprises alternating residues of 3,9-diethyl-3,9-2,4,8,10-tetraoxaspiro[5.5]undecane-3,9-diyl:

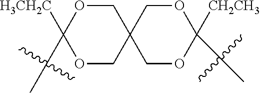

and a diol-ate residue of triethylene glycol or of triethylene glycol diglycolide prepared by reacting triethylene glycol with from 0.5 to 10 molar equivalents of glycolide at about 100° C.-200° C. for about 12 hours to 48 hours, wherein the mole percentage of glycolide-containing subunits in the polyorthoester is from about 0.1 to about 25 mole percent, and the polyorthoester has a molecular weight of about 1000 Da to 10,000 Da.

In one embodiment of a composition or method as provided herein, the 5-HT3 receptor antagonist is granisetron, palonosetron, ondanestron or a pharmaceutically acceptable salt thereof.

In yet another embodiment of a composition or method as provided herein, the granisetron is in the form of a free base. In yet an alternative embodiment, the granisetron is in the form of an acid addition salt.

A further embodiment, the granisetron or ondanestron is in the form of a solid having a particle size of less than 100 microns.

In one embodiment, the semi-solid comprises about 1-10 wt % granisetron. In another embodiment, the semi-solid composition comprises about 1-5 wt %, 2-3 wt %, 3-5 wt %, or 2 wt %, 3 wt %, 4 wt %, or 5 wt % of granisetron.

In one embodiment, the semi-solid comprises about 1-10 wt % ondansetron. In yet a more particular embodiment, the semi-solid composition comprises about 1-10 wt % or 2 wt %, 3 wt %, 4 wt %, 5 wt %, 6 wt %, 7 wt %, 8 wt %, 9 wt % or 10 wt % of ondansetron.

In one embodiment, the semi-solid pharmaceutical composition comprises about 76 wt % polyorthoester, about 22 wt % polyorthoester compatible liquid excipient and about 2 wt % of the 5-HT3 receptor antagonist.

In one embodiment, the polyorthoester has a molecular weight of about 6,500 Da.

In one embodiment, the semi-solid pharmaceutical composition is stable upon irradiation or sterilization.

In one embodiment, the semi-solid further comprises an NK-1 receptor antagonist. In another embodiment, the semi-solid further comprises about 1-25 wt % or 10-20, or 10 wt %, 11 wt %, 12 wt %, 13 wt %, 14 wt %, 15 wt %, 16 wt %, 17 wt %, 18 wt %, 19 wt %, or 20 wt % of the NK-1 receptor antagonist.

In one embodiment, the NK-1 receptor antagonist is aprepitant, fosaprepitant, or a pharmaceutically acceptable salt thereof.

In one embodiment, the semi-solid further comprises about 10-25 wt %, 10-20 wt % or 10 wt %, 11 wt %, 12 wt %, 13 wt %, 14 wt %, 15 wt %, 16 wt %, 17 wt %, 18 wt %, 19 wt %, or 20 wt % aprepitant or a pharmaceutically acceptable salt thereof. In another embodiment, the semi-solid further comprises about 10-25 wt %, 10-20 wt % or 10 wt %, 11 wt %, 12 wt %, 13 wt %, 14 wt %, 15 wt %, 16 wt %, 17 wt %, 18 wt %, 19 wt %, or 20 wt % fosaprepitant or a pharmaceutically acceptable salt thereof.

In one embodiment, the semi-solid pharmaceutical composition is capable of being dispensed from a 16-25 gauge, 16-22 gauge, 18 gauge, 19 gauge, 10 gauge, 21 gauge or 22 gauge needle.

In one embodiment, the semi-solid pharmaceutical composition comprises the 5-HT3 receptor antagonist and the NK-1 receptor antagonist and the composition is effective to release the 5-HT3 and NK-1 receptor antagonists in a sustained and controlled manner after administration. In another embodiment, the sustained and controlled manner of release occurs over a time period of about 1 day to 10 days, 1 day to 7 days, 2 days, 3 days, 4 days, 5 days, 6 days, 7 days, 8 days, 9 days, or 10 days.

In one aspect, a kit comprising a pharmaceutical dosage form comprising a 5-HT3 receptor antagonist and a NK-1 receptor antagonist is provided.

In one aspect, a kit comprising a dosage form comprising a 5-HT3 receptor antagonist and a NK-1 receptor antagonist is provided.

In one embodiment, the kit comprises a first dosage form comprising a selective 5-HT3 receptor antagonist and second dosage form comprising a NK-1 receptor antagonist. In another embodiment, the first and/or second dosage form is in the form of a single dose vial or a prefilled syringe. In yet another embodiment, the first and/or second dosage form is in the form of a multiple dose (e.g., a multiple dose vial). The dosage forms may be packaged together with other optional components such as needles, injection aids, alcohol swabs, other agents.

In one embodiment, the pharmaceutical composition is a semi-solid composition comprising a 5-HT3 receptor antagonist and a NK-1 receptor antagonist.

In one embodiment, the NK-1 receptor antagonist is aprepitant.

In one embodiment, the NK-1 receptor antagonist is fosaprepitant or a pharmaceutically acceptable salt thereof (e.g., fosaprepitant dimeglumine).

In one embodiment the 5-HT3 receptor antagonist is granisetron or a pharmaceutically acceptable salt thereof.

In one embodiment the 5-HT3 receptor antagonist is ondansetron or a pharmaceutically acceptable salt thereof.

In one aspect, a method for preventing, treating, reducing or alleviating a disease or disorder is provided comprising administering a pharmaceutical composition which comprises a 5-HT3 receptor antagonist and a NK-1 receptor antagonist. In one embodiment, the administering comprises subcutaneously injecting the pharmaceutical composition.

In one embodiment, a method for preventing, treating, reducing or alleviating a disease or disorder is provided comprising administering a first pharmaceutical composition which comprises a 5-HT3 receptor antagonist and administering a second pharmaceutical composition which comprises a NK-1 receptor antagonist. In another embodiment, the administering comprises subcutaneously administering the first pharmaceutical composition and subcutaneously administering the second pharmaceutical formulation.

In one embodiment, the administering of the first pharmaceutical composition is simultaneous with the administering of the second pharmaceutical formulation. In the methods of the present disclosure, the two agents are administered simultaneously or sequentially or administered such that there is an overlap of the dosing interval of the two agents.

In one embodiment, the disease or disorder is acute and delayed chemotherapy-induced nausea and vomiting (CINV) in a subject. In another embodiment, the CINV follows a course of emetogenic chemotherapy in the subject. In yet another embodiment, the disease or disorder is radiation-induced nausea and vomiting (RINV), post-operative nausea and vomiting (PONV), pruritus, alcohol dependence, osteoarthritis pain, depression and/or anxiety, post-traumatic stress disorder (PTSD), urinary tract infection, or motion sickness.

In one embodiment, the method is for treating CINV associated with highly emetogenic chemotherapy. In another embodiment, the method is for treating CINV associated with moderately emetogenic chemotherapy.

In one embodiment, the administering comprises subcutaneous injection of the pharmaceutical composition comprising the 5-HT3 receptor antagonist and the NK-1 receptor antagonist.

In one embodiment, the administering prolongs the antiemetic activity compared to an immediate release dosage form. In another embodiment, the administering provides sustained and controlled release of a therapeutically effective amount of a selective 5-HT3 receptor antagonist and an NK-1 receptor antagonist to minimize the side effects of nausea and/or emesis associated with other pharmacological agents (e.g., chemotherapeutic agents).

In a further embodiment, there is provided a pharmaceutical composition for the treatment or prevention of emesis comprising a selective 5-HT3 receptor antagonist and an NK-1 receptor antagonist together with at least one pharmaceutically acceptable carrier or excipient.

In one embodiment, the NK-1 receptor antagonist is aprepitant.

In one embodiment, the NK-1 receptor antagonist is fosaprepitant or a pharmaceutically acceptable salt thereof (e.g., fosaprepitant dimeglumine).

In one embodiment the 5-HT3 receptor antagonist is granisetron or a pharmaceutically acceptable salt thereof.

In one embodiment the 5-HT3 receptor antagonist is ondansetron or a pharmaceutically acceptable salt thereof.

The agents can be administered by any route of administration, e.g., by the oral (e.g., buccal, sublingual, solid oral dosage form), parenteral (e.g., intravenous, intramuscular, subcutaneous), topical (e.g., transdermal), rectal or nasal route.

In certain embodiments, the 5-HT3 receptor antagonist is administered subcutaneously and the NK-1 receptor antagonist is administered orally.

In other embodiments, the 5-HT3 receptor antagonist is administered transdermally and the NK-1 receptor antagonist is administered orally.

In other embodiments, both agents are administered orally, subcutaneously or transdermally. In other embodiments, the disclosure is directed to a single active agent dosage form (e.g., for subcutaneous administration) comprising aprepitant or a pharmaceutically acceptable salt thereof and a polyorthoester and methods of treatment thereof (e.g., nausea and vomiting).

In other embodiments, the disclosure is directed to a single active agent dosage form (e.g., for subcutaneous administration) comprising fosaprepitant or a pharmaceutically acceptable salt thereof (e.g., fosaprepitant dimeglumine) and a polyorthoester and methods of treatment thereof (e.g., nausea and vomiting).

Additional embodiments of the present compositions and methods, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE FIGURES

FIG. 1 is a graph demonstrating the in vitro release of granisetron and fosaprepitant from an exemplary semi-solid composition comprising the same as described in greater detail in Example 6.

FIG. 2 is a graph illustrating pharmacokinetic data for an exemplary semi-solid composition comprising granisetron and fosaprepitant administered to dogs as described in greater detail in Example 7.

FIG. 3 is a graph illustrating the in vitro release of granisetron and aprepitant from an exemplary semi-solid composition comprising the same as described in greater detail in Example 1.

FIG. 4 is a graph illustrating the in vitro release of aprepitant from an exemplary semi-solid composition comprising the same as described in greater detail in Example 3.

### DETAILED DESCRIPTION

### Definitions

Unless defined otherwise in this specification, all technical and scientific terms are used herein according to their conventional definitions as they are commonly used and understood by those of ordinary skill in the art of synthetic chemistry, pharmacology and medicine.

“Active agent” includes any compound or mixture of compounds which produces a pharmacologic, and often beneficial or useful result. Active agents are distinguishable from such components as vehicles, carriers, diluents, lubricants, binders and other formulating aids, and encapsulating or otherwise protective components. Examples of active agents and their pharmaceutically acceptable salts are pharmaceutical, agricultural or cosmetic agents.

“Basic active agent” means an active agent as defined above wherein the active agent has basic properties or functionalities. Examples include compounds that are Lewis bases having nonbonding pairs of electrons or Bronsted bases. Examples of such as agents include those having an amine or nitrogen containing group. The basic active agent may also include compositions comprising an active agent that has basic properties or functionalities as defined above.

“Biologically active organic compound” means an active agent, as defined above, wherein the active agent is an organic compound.

“Bioerodible” and “bioerodibility” refer to the degradation, disassembly or digestion of the polyorthoester by action of a biological environment, including the action of enzymes, living organisms and most notably physiological pH and temperature. A principal mechanism for bioerosion of the polyorthoesters of the present disclosure is hydrolysis of linkages between and within the units of the polyorthoester.

“Comprising” is an inclusive term interpreted to mean containing, embracing, covering or including the elements listed following the term, but not excluding other unrecited elements.

“Controlled release,” “sustained release,” and similar terms are used to denote a mode of active agent delivery that occurs when the active agent is released from the delivery vehicle at an ascertainable and controllable rate over a period of time, rather than dispersed immediately upon administration. Controlled or sustained release may extend for hours, days or months, and may vary as a function of numerous factors. For the pharmaceutical composition of the present disclosure, the rate of release may depend on the type of the excipient selected and the concentration of the excipient in the composition. Other factors determining the rate of release of an active agent from the present pharmaceutical composition includes particle size, solubility of the active agent, acidity of the medium (either internal or external to the matrix), physiochemical interactions with the matrix, and physical and chemical properties of the active agent within the dosage form.

“Molecular mass” in the context of a polyorthoester, refers to the nominal average molecular mass of a polymer, typically determined by size exclusion chromatography, light scattering techniques, or velocity. Molecular weight can be expressed as either a number-average molecular weight or a weight-average molecular weight. Unless otherwise indicated, all references to molecular weight herein refer to the weight-average molecular weight. Both molecular weight determinations, number-average and weight-average, can be measured using gel permeation chromatographic or other liquid chromatographic techniques. Other methods for measuring molecular weight values can also be used, such as the measurement of colligative properties (e.g., freezing-point depression, boiling-point elevation, or osmotic pressure) to determine number-average molecular weight or the use of light scattering techniques, ultracentrifugation or viscometry to determine weight-average molecular weight. The polymers of the invention are typically polydisperse (i.e., number-average molecular weight and weight-average molecular weight of the polymers are not equal), possessing low polydispersity values such as less than about 1.2, less than about 1.15, less than about 1.10, less than about 1.05, and less than about 1.03.

In embodiments comprising polyorthoesters, the rate of release is determined at least in part by the rate of hydrolysis of the linkages between and within the units of the polyorthoester. The rate of hydrolysis in turn may be controlled by the composition of the polyorthoester and the number of hydrolyzable bonds in the polyorthoester.

“Delivery vehicle” denotes a composition which has the functions including transporting an active agent to a site of interest, controlling the rate of access to, or release of, the active agent by sequestration or other means, and facilitating the application of the agent to the region where its activity is needed.

“Matrix” denotes the physical structure of a polymer-based composition (e.g., a polyorthoester) which essentially retains the active agent in a manner preventing release of the agent until the polymer erodes or decomposes. “Polyorthoester-compatible” refers to the properties of an excipient which, when mixed with a polyorthoester, forms a single phase and does not cause any physical or chemical changes to the polyorthoester.

“Pro-drug” denotes a pharmacologically inactive or less active form of a compound which is changed or metabolized in vivo, e.g., by biological fluids or enzymes, by a subject after administration into a pharmacologically active or more active form of the compound in order to produce the desired pharmacological effect. Prodrugs of a compound can be prepared by modifying one or more functional group(s) present in the compound in such a way that the modification(s) are cleaved or altered in vivo to release the parent compound. Prodrugs include compounds wherein a hydroxy, amino, sulfhydryl, carboxy or carbonyl group in a compound is bonded to any group that can be cleaved in vivo to regenerate the free hydroxyl, amino, sulfhydryl, carboxy or carbonyl group respectively. Examples of prodrugs include, but are not limited to, esters (e.g. acetate, dialkylaminoacetates, formates, phosphates, sulfates and benzoate derivatives) and carbamates of hydroxy functional groups (e.g. N,N-dimethylcarbonyl), esters of carboxyl functional groups (e.g. ethyl esters, morpholinoethanol esters), N-acyl derivatives (e.g. N-acetyl), N-Mannich bases, Schiff bases and enaminones of amino functional groups, oximes, acetals, ketals, and enol esters of ketones and aldehyde functional groups in a compound, and the like. An exemplary prodrug is fosaprepitant, a phosphoryl prodrug form of aprepitant.

“Semi-solid” denotes the mechano-physical state of a material that is flowable under moderate stress. More specifically, the semi-solid material may have a viscosity between about 10,000 centipoise (cp) and 3,000,000 cp, especially between about 30,000 cp and 500,000 cp when measured as a 2 wt % solution at 25° C. Preferably the formulation is syringable or injectable, meaning that it can readily be dispensed from a conventional tube of the kind well known for topical or ophthalmic formulations, from a needleless syringe, or from a syringe with a 16 gauge or smaller needle, such as 16-25 gauge.

A “therapeutically effective amount” means the amount that, when administered to an animal for treating a disease, is sufficient to effect treatment for that disease.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). For the purposes of this embodiments described herein, a “disease” includes pain.

### Compositions and Methods of Use

The goal of antiemetic therapy is the complete prevention of CINV. Currently, there are two major categories of drugs with the highest therapeutic index for the management of CINV. These are: 5-HT3 receptor antagonists (e.g., granisetron and ondansetron) and NK1 receptor antagonists (e.g., aprepitant and fosaprepitant). Unfortunately, as treatment with any single agent has not been shown to provide complete prevention of CINV for all or almost all patients in need, studies are in progress to identify combinations which can provide the desired efficacy. While it is desirable to provide prophylactic treatment to prevent any onset of nausea and/or vomiting, the present disclosure is understood to also include treatment after onset of symptoms such as nausea and/or vomiting.

### Pharmaceutical Compositions

Provided herein are pharmaceutical compositions comprising a 5-HT3 receptor antagonist and a NK-1 receptor antagonist to provide combination therapy for CINV, RINV, and other diseases or disorders as noted above. In a preferred embodiment, a semi-solid delivery vehicle is formulated which, when injected subcutaneously, can provide sustained release of one or more active agents to a subject in need thereof. The semi-solid delivery vehicle may contain both the 5-HT3 receptor antagonist and the NK-1 receptor antagonist, wherein the delivery vehicle provides sustained release of both active agents over an extended period of time upon subcutaneous injection of a pharmaceutical composition comprising the delivery vehicle. The composition is formulated such that the delivery vehicle provides sustained or controlled release of one or both active agents to the subject over a time period of about 0.5 h to 240 h, 0.5 h to 120 h, or about 72 h, 120 h, 144 h, 168 h, 192 h, 216 h or 240 h.

There are several drug delivery systems that are suitable for the sustained and controlled release of a selective 5-HT3 receptor antagonist and an NK-1 receptor antagonist in the compositions and methods described herein, as they are particularly tailored to be subcutaneous, such as compositions comprising the semisolid polymers described in U.S. Pat. Nos. 5,968,534, 6,613,335, 6,790,458, all to Heller et al and in US 2007/0264339 to Shah; all of which are incorporated herein by reference. These exemplary semi-solid polyorthoester polymers are generally prepared by condensation reactions between diketene acetals and polyols, preferably diols, to provide polymers having differences in their mechanophysical state and bioerodibility, based upon the selection of the diol component(s), to be explained in greater detail below.

The semi-solid composition is then filled into a syringe optionally with a 16-25 gauge needle, although small needles may be used in some embodiments, and injected into sites that have been determined to be most effective. The semi-solid injectable composition of the present disclosure can be used for controlled delivery of both slightly soluble and soluble antiemetic agents.

As stated above, preferred semisolid polymers are polyorthoesters. Preferred polyorthoesters that can be utilized in the presently disclosed compositions are selected from the group consisting of

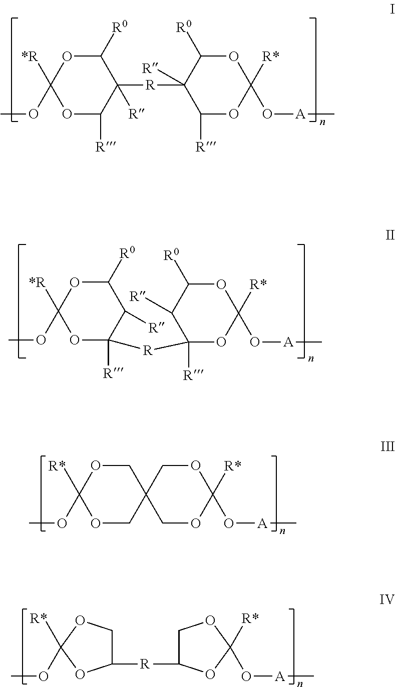

where:
R is a bond, —(CH₂)ₐ—, or —(CH₂)_{b}—O—(CH₂)_{c}—; where a is an integer from 1 to 10, and b and c are independently integers from 1-5; R* is a C₁₋₄ alkyl;
R⁰, R^{II} and R^{III} are each independently H or C₁₋₄ alkyl;
n is an integer of at least 5, for example, from 5 to 1000; and
A is R¹, R², R³, or R⁴, where
R¹ is:

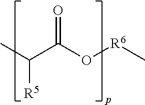

where:
p is an integer of 1 to 20;
R⁵ is hydrogen or C₁₋₄ alkyl; and
R⁶ is:

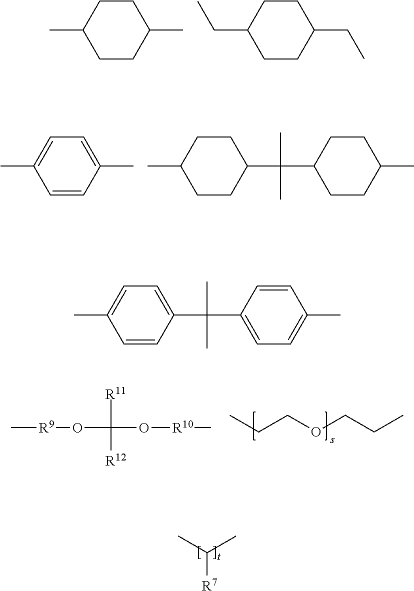

where:
s is an integer of 0 to 30;
t is an integer of 2 to 200; and
R⁷ is hydrogen or C₁₋₄ alkyl;
R² is:

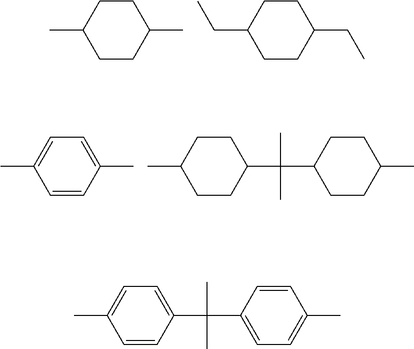

R³ is:

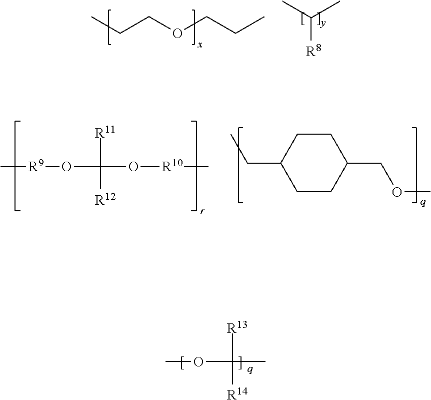

where:
x is an integer of 0 to 100;
y is an integer of 2 to 200;
q is an integer of 2 to 20;
r is an integer of 1 to 20;
R⁸ is hydrogen or C₁₋₄ alkyl;
R⁹ and R¹⁰ are independently C₁₋₁₂ alkylene;
R¹¹ is hydrogen or C₁₋₆ alkyl and R¹² is C₁₋₆ alkyl; or R₁₁ and R₁₂ together are C₃₋₁₀ alkylene; and
R⁴ is the residue of a diol containing at least one functional group independently selected form amide, imide, urea, and urethane groups;
in which at least 0.01 mol percent of the A units are of the formula R¹.

Exemplary polyorthoesters possess a molecular weight of about 1,000 Da to 20,000 Da, for example from 1,000 Da to 10,000 Da or preferably from 1,000 Da to 8,000 Da, or from about 1,500 Da to about 7,000 Da.

Particularly preferred polymers are prepared by reaction of a diketene acetal according to one of the following formulas:

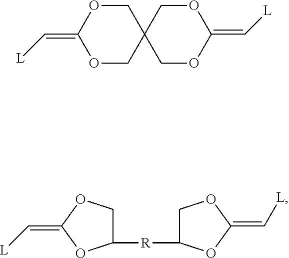

where L is hydrogen or a C₁₋₃ alkyl, and R is as defined above, with a diol according to formula HO—R¹—OH and at least one diol according to the formulae, HO—R²—OH, HO—R³—OH, or HO—R⁴—OH (where R¹, R², R³ and R⁴ are as described above). In the presence of water, the α-hydroxy acid containing subunits are readily hydrolyzed at body temperature and at physiological pH to produce the corresponding hydroxyacids, which can then act as catalysts to control the hydrolysis rate of the polyorthoester without the addition of exogenous acid. Thus, polyorthoesters having a higher mole percentage of α-hydroxy acid containing subunits possess a higher degree of bioerodibility.

Preferred polyorthoesters are those in which the mole percentage of α-hydroxy acid containing subunits is at least about 0.01 mole percent. Exemplary percentages of α-hydroxy acid containing subunits in the polymer are from about 0.01 to about 50 mole percent, preferably from about 0.05 to about 30 mole percent, from about 0.1 to about 25 mole percent. As an illustration, the percentage of α-hydroxy acid containing subunits may be 1, 2, 3, 4, 5, 6, 7, 8, 9, 10, 11, 12, 13, 14, 15, 16, 17, 18, 19, 20, 21, 22, 23, 24, 24, 26, 27, 28, 29 or 30 mol percent, including any and all ranges lying therein, formed by combination of any one lower mole percentage number with any higher mole percentage number.

Exemplary preferred polyorthoesters are those in which R⁵ is hydrogen or methyl; R⁶ is

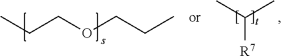

where s is an integer from 0 to 10, e.g., preferably selected from 1, 2, 3, or 4; t is an integer from 2 to 30 particularly selected from 2, 3, 4, 5, 6, 7, 8, 9 and 10; R⁷ is hydrogen or methyl; and R³ is

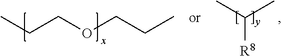

where x is an integer from 0 to 10, e.g., preferably selected from 1, 2, 3, or 4; y is an integer from 2 to 30, particularly selected from 2, 3, 4, 5, 6, 7, 8, 9 and 10; R⁸ is hydrogen or methyl; R⁴ is selected from a residue of an aliphatic diol having from 2-20 carbon atoms (e.g., selected from 2, 3, 4, 5, 6, 7, 8, 9, 10, 11, 12, 13, 14, 15, 16, 17, 18, 19, and 20 carbon atoms), preferably having from 2 to 10 carbon atoms, interrupted by by one or two amide, imide, urea, or urethane groups. Preferably, the proportion of subunits in the polyorthoester in which A is R¹ is from about 0.01-50 mole percent, preferably from about 0.05 to about 30 mole percent, and more preferably from about 0.1 to 25 mole percent. Illustrative and preferred mole percentages include 10, 15, 25 and 25 mole percent of percentage of subunits in the polyorthoester in which A is R¹. In one preferred embodiment, the mole percent is 20. Additionally, typically, the proportion of subunits in which A is R2 is less than 20 percent, preferably less than about 10 percent, and more preferably less than about 5 percent, and the proportion of subunits in which A is R4 is less than 20 percent, preferably less than about 10 percent and more preferably less than 5 percent.

An exemplary and preferred polyorthoester comprises subunits selected from

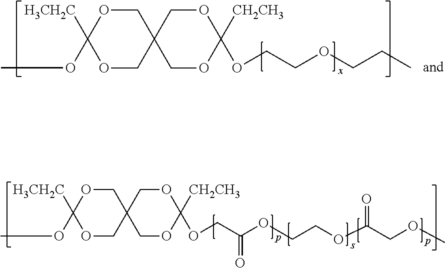

where

x is an integer from 1-4 (e.g., can be selected from 1, 2, 3, and 4)

the total amount of p is an integer from 1-20 (e.g., can be selected from 1, 2, 3, 4, 5, 6, 7, 8, 9, 10, 11, 12, 13, 14, 15, 16, 17, 18, 19, and 20),

s is an integer from 1-4 (e.g., can be selected from 1, 2, 3, and 4),

the mole percentage of α-hydroxyacid containing subunits in the polyorthoester is from about 0.1 to about 25 mole percent, and the polyorthoester has a molecular weight in a range of about 1,000 Da to 10,000 Da.

An exemplary polyorthoester comprises alternating residues of 3,9-diethyl-3,9-2,4,8,10-tetraoxaspiro[5.5]undecane-3,9-diyl:

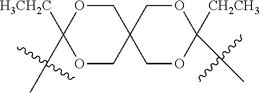

and a diol-ate residue of triethylene glycol or of triethylene glycol diglycolide prepared by reacting triethylene glycol with from 0.5 to 10 molar equivalents of glycolide at about 100-200° C. for about 12 hours to 48 hours. Typically, the mole percentage of glycolide-containing subunits in the polyorthoester is from about 0.1 to about 25 mole percent, and the polyorthoester has a molecular weight of about 1,000 Da to 10,000 Da.

As an example, polyorthoesters such as those described above are prepared by reacting an illustrative diketene acetal, 3,9-di(ethylidene)-2,4,8,10-tetraoxaspiro[5.5]undecane (DETOSU),

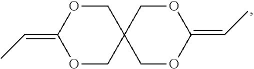

with one or more diols as described above, e.g., triethylene glycol (TEG) and triethylene glycol diglycolide (TEGdiGL). Diols such as triethylene diglycolide or triethylene monoglycolide, or the like, are prepared as described in U.S. Pat. No. 5,968,543, e.g., by reacting triethylene glycol and glycolide under anhydrous conditions to form the desired product. For example, a diol of the formula HO—R¹—OH comprising a polyester moiety may be prepared by reacting a diol of the formula HO—R⁶—OH with between 0.5 and 10 molar equivalents of a cyclic diester of an α-hydroxy acid such as lactide or glycolide, and allowing the reaction to proceed at 100-200° C. for about 12 hours to about 48 hours. Suitable solvents for the reaction include organic solvents such as dimethylacetamide, dimethyl sulfoxide, dimethylformamide, acetonitrile, pyrrolidone, tetrahydrofuran, and methylbutyl ether. Although the diol product is generally referred to herein as a discrete and simplified entity, e.g., TEG diglycolide (and products such as TEG diglycolide), it will be understood by those of skill in the art that due to the reactive nature of the reactants, e.g., ring opening of the glycolide, the diol is actually a complex mixture resulting from the reaction, such that the term, TEG diglycolide, generally refers to the average or overall nature of the product. In a preferred embodiment, the polyorthoester is prepared by reacting DETOSU, triethylene glycol, and triethylene glycol diglycolide in the following molar ratios: 90/80/20.

A preferred polyorthoester is prepared by reacting 3,9-di(ethylidene)-2,4,8,10-tetraoxaspiro[5.5]undecane (DETOSU) with the diols, triethylene glycol and triethylene glycol diglycolide. Generally, the polyorthoester is prepared by reacting DETOSU:TEG:TEG-diGL a molar ratio of 90:80:20. In a representative reaction, carried out under anhydrous conditions, DETOSU and triethylene glycol are dissolved in an anhydrous solvent such as tetrahydrofuran. Triethylene glycol diglycolide is introduced into a separate vessel, dissolved in a suitable solvent such as anhydrous tetrahydrofuran, and the resulting solution is then added to the DETOSU-TEG solution to initiate the polymerization. Generally, the reaction mixture will come to a boil as the polymerization reaction proceeds. The resulting solution is then cooled to ambient (room) temperature, concentrated under vacuum, optionally at an elevated temperature (e.g., 50-80° C.), to provide a semi-solid. In a preferred embodiment, the resulting polyorthoester has a molecular weight of about 6,500 daltons.

Thus, in a particularly preferred embodiment, the polyorthoester comprises about 20 mole percent R¹, where R¹ is triethylene glycol diglycolide, and 80 mole percent R³, where R³ is triethylene glycol.

The semi-solid compositions provided herein may also contain one or more excipients. Preferably, the excipient is a pharmaceutically-acceptable polyorthoester compatible liquid excipient. Such excipients are liquid at room temperature and are readily miscible with polyorthoesters. Exemplary polyorthoester compatible liquid excipients include polyethylene glycol having a molecular weight between about 200 Da and 4,000 Da, or a polyethylene glycol derivative or co-polymer having a molecular weight between about 200 Da and 4,000 Da, e.g., an end-capped PEG such as monomethoxypolyethylene glycol, or a mono-, di- or triglyceride of a C2-19 aliphatic carboxylic acid or a mixture of such acids, alkoxylated tetrahydrofurfuryl alcohols and their C1-C4 alkyl ethers, dimethyl sulfoxide (DMSO), and C2-19 aliphatic carboxylic acid esters, or the like. A preferred excipient is monomethoxy-PEG, having a molecular weight selected from 400, 450, 500, 550, 600 and 650.

The semi-solid composition, sometimes referred to as a delivery vehicle, is typically prepared by mixing or blending the polyorthoester and the polyorthoester-compatible liquid. The mixing or blending can be performed by any suitable method, generally at a temperature less than about 50° C., e.g., at room temperature, although in certain instances, depending upon the nature of the materials, mixing or blending may be carried out at higher temperatures, e.g., from about 25 to 100° C. The mixing or blending is generally carried out in the absence of solvents, to obtain a homogeneous, flowable and non-tacky semi-solid formulation at room temperature.

The 5-HT3 receptor antagonist and the NK-1 receptor antagonist, if themselves liquids or semi-solids, may be mixed with the semi-solid composition in the same manner as which it was formed, i.e., by conventional blending. The 5-HT3 receptor antagonist and the NK-1 receptor antagonist may be blended in the same semi-solid composition, or in separate compositions. The blending is generally carried out in a fashion suitable to obtain a homogeneous distribution of the components in the formulation, i.e., by mixing the components in any order necessary to achieve homogeneity. In cases in which the active agent, i.e., the 5-HT3 receptor antagonist and/or the NK-1 receptor antagonist, is a solid, which is often the case, it is preferred that the particle size is sufficiently small (e.g., 1-100 microns, or preferably, from 5-50 microns), to provide a resulting composition that is smooth. In many instances, i.e., unless the active agent is provided in micron-sized powder form, the active agent is milled into fine particles preferably less than 100 microns and sieved before mixing with the other semi-solid components. The active agent may be mixed with the semi-solid composition that has already been formed or can be mixed together with the polyorthoester and polyorthoester-compatible liquid to form the final semi-solid composition. The components, including the active agent, may be mixed in any order to achieve a homogeneous composition.

A preferred semi-solid composition contains a polyorthoester, polyethylene glycol, polyethylene glycol monomethylether 550 (also referred to as mPEG or monomethoxy PEG), and at least one active agent that is either a 5-HT3 receptor antagonist or an NK-1 receptor antagonist. The polyorthoester is prepared from DETOSU:TEG:TEG-diGL, at a molar ratio of 90:80:20). The relative concentrations of the components of the semi-solid composition will vary depending upon the amount of active agent(s), polyorthoester, and polyorthoester-compatible liquid. The weight percent of the polyorthoester compatible liquid can range from about 10-50 weight percent, or from about 10-40 weight percent, or from 10-30 weight percent, or from 10-25 weight percent. Exemplary amounts are about 10, 12, 15, 20, 25, 30, 35, 40, 45 or 50 weight percent of the polyorthoester-compatible liquid such as mPEG 550 or any other suitable polyorthoester-compatible liquid as described previously in the final semi-solid composition. Preferably, the amount of polyorthoester-compatible liquid is selected from 10, 11, 12, 13, 14, 15, 16, 17, 18, 19, 20, 21, 22, 23, 24, 25, 26, 27, 28, 29, and 30 weight percent. The amount of the 5-HT3 receptor antagonist, (e.g., granisetron, palonosetron, or ondanestron, optionally in the form of its acid salt), will generally range from about 1-10 weight percent. Illustrative amounts further include from about 1-5 weight percent of the 5-HT3 receptor antagonist, or about 1, 2, 3, 4, or 5 weight percent of the 5-HT3 receptor antagonist as described above. In a preferred embodiment, the 5-HT3 receptor antagonist is granisetron. The amount of the NK-1 receptor antagonist in the semi-solid formulation is generally from about 1-25 weight percent, preferably from about 10-20 weight percent in the final semi-solid composition. In certain embodiments, the weight percentage of the NK-1 receptor antagonist is selected from 10, 11, 12, 13, 14, 15, 16, 17, 18, 19 and 20 weight percent. Preferred NK-1 receptor antagonists include aprepitant and fosaprepitant, optionally in the form of a pharmaceutically acceptable salt. Exemplary semi-solid compositions may comprise any one of the following relative weight percentages of components: (i) from about 10-40 weight percent polyorthoester-compatible liquid, about 1-10 weight percent 5-HT3 receptor antagonist, and/or about 1-25 weight percent NK-1 receptor antagonist, with the remainder being polyorthoester and/or additional optional excipients and/or additives; (ii) from about 10-40 weight percent polyorthoester-compatible liquid, about 1-15 weight percent 5-HT3 receptor antagonist, and/or about 1-20 weight percent NK-1 receptor antagonist, (iii) from about 10-30 weight percent polyorthoester-compatible liquid, about 1-10 weight percent 5-HT3 receptor antagonist, and/or about 1-25 weight percent NK-1 receptor antagonist, (iv) from about 10-30 weight percent polyorthoester-compatible liquid, about 1-5 weight percent 5-HT3 receptor antagonist, and/or about 1-20 weight percent NK-1 receptor antagonist.

Particular embodiments of the semi-solid compositions will now be provided.

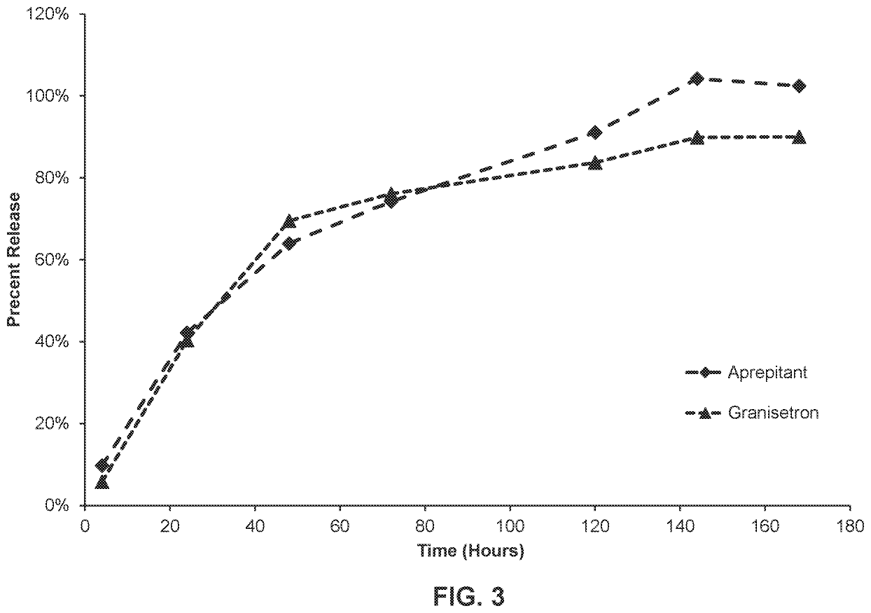

In a first embodiment, 375 mg of aprepitant was dissolved in 1.3 g of a polyorthoester-compatible liquid excipient, dimethyl sulfoxide, and heated until completely dissolved. 100 mg of granisetron was added to the mixture and heated until the granisetron and the aprepitant were completely dissolved. The polyorthoester (having a molar ratio of DETOSU:TEG:TEG-diGL of about 90:80:20) was brought to approximately 80° C. and mixed into the solution comprising the granisetron and aprepitant until completely homogenous to provide a semi-solid formulation comprising the illustrative 5-HT3 receptor antagonist and NK-1 receptor antagonist, granisetron and aprepitant, respectively. In a further embodiment, the formulation was weighed into vials and filled with a phosphate buffered saline solution and 2% CTAB (cetrimonium bromide). The vials were then stored at 37° C. Aliquots were taken daily and analyzed by HPLC to monitor for the release of granisetron and aprepitant from the formulation. See FIG. 3. As shown in FIG. 3, in vitro release of each of the active agents proceeded in a linear fashion up to about 48 hours, with about 65% of each active agent released over this time period. After about 120 hours, about 90% of the aprepipant was released from the formulation, while about 80% of granisetron had been released over the same time frame. Essentially all of the aprepitant is released from the formulation at about 144 hours, while about 90% of the granisetron is released over the same duration.

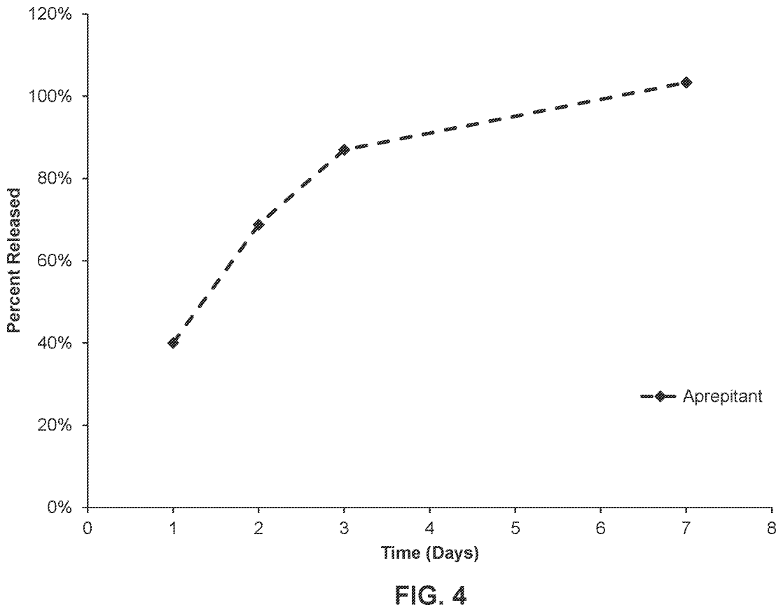

In a second embodiment, 750 mg of aprepitant was dissolved in 2.3 g of a polyorthoester-compatible liquid excipient, dimethyl sulfoxide, and heated until completely dissolved. The polyorthoester (80:20 TEG-TEG diglycolide diol molar ratio) was brought to approximately 80° C. and mixed into the solution comprised of aprepitant until completely homogenous. In a further embodiment, the formulation was weighed into vials and filled with a phosphate buffered saline solution and 2% CTAB. The vials were then stored at 37° C. and analyzed by HPLC to monitor for the release of aprepitant from the formulation (see FIG. 4). As shown in FIG. 4, in vitro release of aprepitant proceeded in a linear fashion up to about 72 hours.

In a third embodiment, a formulation containing 150 mg of fosaprepitant (a pro-drug form of aprepitant) dissolved in 250 mg of a polyorthoester-compatible liquid excipient, dimethyl sulfoxide, was weighed into an appropriately sized container. The mixture was heated until completely dissolved. The polyorthoester described in Example 1 (80:20 TEG-TEG diglycolide diol ratio) was brought to approximately 80° C. and mixed into the solution of fosaprepitant until completely homogenous.

In a fourth embodiment, 500 mg of a polyorthoester-compatible liquid excipient, dimethyl sulfoxide, was weighed into an appropriately sized container containing 40 mg of granisetron. The mixture was heated until dissolved and then 300 mg of fosaprepitant (a pro-drug form of aprepitant) was added and completely dissolved to provide a solution comprising granisetron and fosaprepitant. The polyorthoester described in Example 1 (80:20 TEG-TEG diglycolide diol ratio) was brought to approximately 80° C. and mixed into the solution comprising the granisetron and fosaprepitant until completely homogenous.

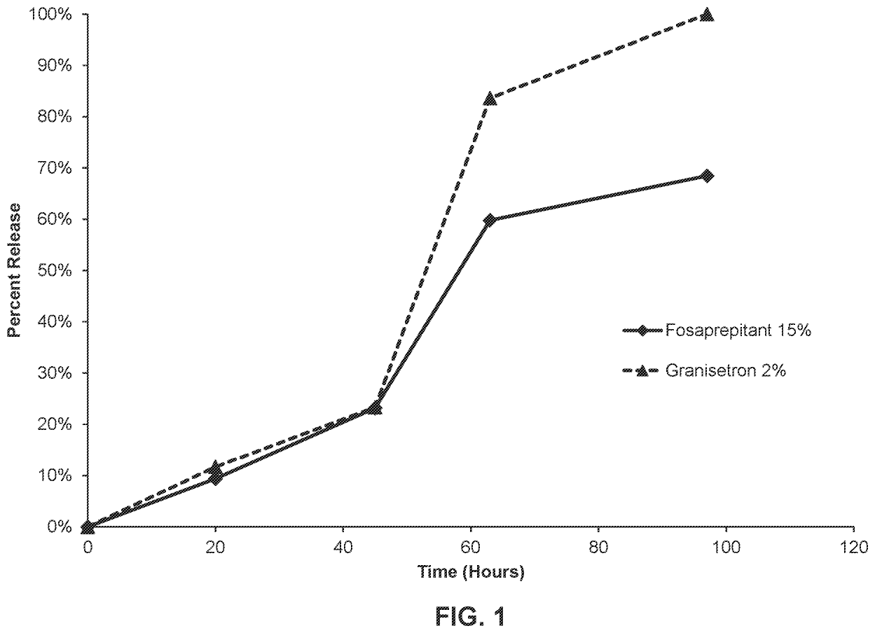

In a fifth embodiment, a formulation as described in Example 6 was weighed into vials and filled with a phosphate buffered saline solution. The vials were then stored at 37° C. and analyzed by HPLC to monitor for the release of granisetron and fosaprepitant from the formulation (see FIG. 1). As shown in FIG. 1, in vitro release of each of the active agents proceeded in a linear fashion up to about 45 hours, with about 25% of each active agent released over this time period. After about 60 hours, nearly 60% of the fosaprepitant (actually, the aprepipant) was released from the formulation, while about 80% of granisetron had been released over the same time frame. Nearly all of the granisetron is released from the formulation at about 100 hours, while about 65% of the aprepitant is released over the same duration. This data demonstrates that both active agents are released in a sustained fashion over time from the exemplary semi-solid formulation.

In a sixth embodiment, a subcutaneous injection consisting of 0.5 gm of the formulation described in Example 5 was administered to five dogs. Each injection contained 20 mg/gm of granisetron and 150 mg/gm fosaprepitant. The syringes were stored at 2-8 C and brought to room temperature 1 hour before administration. Each dog received a single subcutaneous injection of the entire contents of 1 syringe (0.5 gm) over the dorsal lumbar muscle on Day 1. Blood was drawn from all dogs at the following time points: T=0, (immediately prior to drug administration), 1, 6, 12, 24, 48, 72, 96, 120, 144, and 168 hours post administration. The plasma samples were analyzed by HPLC for aprepitant and granisetron

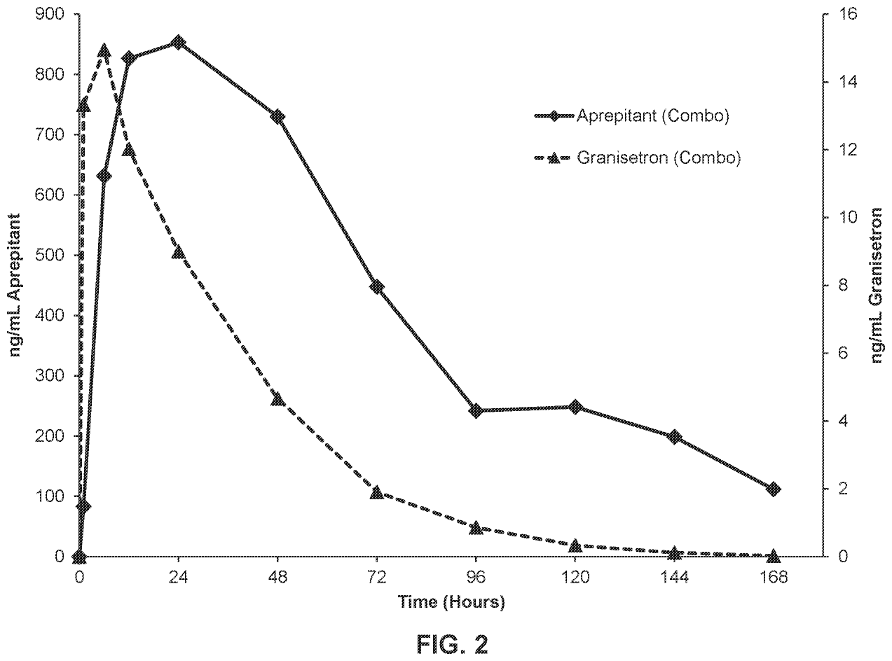

As can be seen from the graph, the semi-solid formulation is effective to provide sustained release of both granisetron and aprepitant In looking at FIG. 2, it can be seen Cmax for granisetron is reached at about 6 hours, while Cmax for aprepitant is reached at about 24 hours. The areas under both curves indicate good bioavailability for both drugs upon administration from a semi-solid dosage form such as described herein.

In certain embodiments, the present disclosure includes the pharmaceutical compositions and methods disclosed and published in U.S. Patent Application Publication Nos. 2005/0042194; 2007/0264338; 2007/0265329 and 2010/0152227, which are all hereby incorporated by reference in their entireties for all purposes. These compositions can be modified to include a 5-HT3 receptor and an NK-1 receptor antagonist in the same dosage formulation or in different dosage formulations.

In one embodiment, provided is a pharmaceutical composition for the sustained or controlled release of an effective amount of a selective 5-hydroxytryptamine 3 (5-HT3) receptor antagonist and an NK-1 receptor antagonist. It is understood that such a combination pharmaceutical composition may comprise the 5-HT3 receptor antagonist in one pharmaceutical formulation or delivery vehicle and the NK-1 receptor antagonist in a separate pharmaceutical formulation or delivery vehicle. Accordingly, the administration of the two separate formulations can be carried out concurrently, or in an overlapping matter wherein administration of the 5-HT3 receptor antagonist is initiated about 1 minute (min), 5 minutes, 10 min or 15 min prior to initiating administration of the NK-1 receptor antagonist. Alternatively, administration of the NK-1 receptor antagonist is initiated about 1 minute (min), 5 minutes, 10 min or 15 min prior to initiating administration of the 5-HT3 receptor antagonist. In one embodiment, administration of the individual 5-HT3 and NK-1 receptor antagonist formulations to the patient (i.e., subcutaneous injection formulation) is done in at two different times.

Administration of the pharmaceutical compositions described herein to a subject may provide simultaneous administration of both antagonists. for the prevention, reduction or alleviation of acute and delayed chemotherapy-induced nausea and vomiting (CINV) following a course of emetogenic chemotherapy, wherein the composition is administered by subcutaneous injection, the composition comprising a 5-HT3 receptor antagonist, an NK-1 receptor antagonist, and a delivery vehicle.

The compositions described herein are useful for the treatment or prevention of emesis in a subject, by administering the composition for the sustained and controlled release of an effective amount of a selective 5-hydroxytryptamine 3 (5-HT3) receptor antagonist and an NK-1 receptor antagonist to minimize the side effects of nausea and/or emesis associated with other pharmacological agents. Also provided is a method of using the herein described pharmaceutical compositions for the prevention, reduction or alleviation of acute and delayed chemotherapy-induced nausea and vomiting.

Exemplary compositions, e.g., for the treatment or prevention of emesis comprise a selective 5-HT3 receptor antagonist and an NK-1 receptor antagonist together with at least one pharmaceutically acceptable carrier or excipient. In an exemplary composition, the NK-1 receptor antagonist is aprepitant or a pharmaceutically acceptable salt thereof. In yet another exemplary composition, the NK-1 receptor antagonist is fosaprepitant or a pharmaceutically acceptable salt thereof. Additional exemplary compositions include those in which the 5-HT3 receptor antagonist is granisetron or a pharmaceutically acceptable salt thereof, or the 5-HT3 receptor antagonist is ondansetron or a pharmaceutically acceptable salt thereof.

### Antiemetics

As used herein, the term “emesis” includes nausea and vomiting. The 5-HT3 receptor antagonists in the compositions of the present disclosure are beneficial in and are contemplated for use in the therapy of acute, delayed or anticipatory emesis, including emesis induced by chemotherapy, radiation, toxins, viral or bacterial infections, pregnancy, vestibular disorders (e.g. motion sickness, vertigo, dizziness and Meniere's disease), surgery, migraine, and variations in intracranial pressure. The 5-HT3 antagonists of use as presently disclosed are of particular benefit in the therapy of emesis induced by radiation and/or by chemotherapy, for example during the treatment of cancer, or radiation sickness; and in the treatment of post-operative nausea and vomiting.

Suitable 5-HT3 antagonists include, but are not limited to, metoclopramide, ondansetron, granisetron, tropisetron, palonosetron, and dolasetron, including all pharmaceutically acceptable salts thereof. Currently marketed anti-emetics which have 5-HT3 receptor antagonists include SANCUSO™ (granisetron hydrochloride) and ZOFRAN™ ODT (ondansetron), ALOXI™ (palonosetron hydrochloride), ANZEMET™ (dolasetron mesylate), NAVOBAN (tropisetron), and IRIBO (ramosetron). It is envisioned that the presently disclosed semi-solid pharmaceutical formulations may comprise any one or more of these active agents alone or in combination with a NK-1 receptor antagonist.

The 5-HT3 receptor antagonists dosage forms (e.g., semi-solid injectables) of the present disclosure are beneficial in the therapy of emesis induced by antineoplastic (cytotoxic) agents including those routinely used in cancer chemotherapy, and emesis induced by other pharmacological agents, for example, alpha-2 adrenoceptor antagonists, such as yohimbine, MK-912 and MK-467, and type IV cyclic nucleotide phosphodiesterase (PDE4) inhibitors, such as RS14203, CT-2450 and rolipram.

Particular examples of chemotherapeutic agents are described, for example, by D. J. Stewart in Nausea and Vomiting: Recent Research and Clinical Advances, ed. J. Kucharczyk et al., CRC Press Inc., Boca Raton, Fla., USA, 1991, pages 177-203, see page 188. Examples of commonly used chemotherapeutic agents include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin and chlorambucil (see R. J. Gralle et al. in Cancer Treatment Reports, 1984, 68, 163-172)

In embodiments comprising a semi-solid injectable dosage form described herein, the dosage form may comprise a 5-HT3 receptor antagonist incorporated into a polyorthoester delivery vehicle such as those described above. The concentration of the 5-HT3 receptor antagonist in the composition may vary from about 1 wt % to 10 wt %, 2 wt % to 8 wt %, 2 wt % to 5 wt %, 2 wt % to 3 wt %, or 1 wt % to 5 wt %, and may be about 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.1 wt %, 2.2 wt %, 2.3 wt %, 2.4 wt %, 2.5 wt %, 2.6 wt %, 2.7 wt %, 2.8 wt %, 2.9 wt %, 3 wt %, 3.1 wt %, 3.2 wt %, 3.3 wt %, 3.4 wt %, 3.5 wt %, 3.6 wt %, 3.7 wt %, 3.8 wt %, 3.9 wt %, 4 wt %, 4.1 wt %, 4.2 wt %, 4.3 wt %, 4.4 wt %, 4.5 wt %, 4.6 wt %, 4.7 wt %, 4.8 wt %, 4.9 wt %, 5 wt %, 5.1 wt %, 5.2 wt %, 5.3 wt %, 5.4 wt %, 5.5 wt %, 5.6 wt %, 5.7 wt %, 5.8 wt %, 5.9 wt %, 6 wt %, 6.1 wt %, 6.2 wt %, 6.3 wt %, 6.4 wt %, 6.5 wt %, 6.6 wt %, 6.7 wt %, 6.8 wt %, 6.9 wt %, 7 wt %, 7.1 wt %, 7.2 wt %, 7.3 wt %, 7.4 wt %, 7.5 wt %, 7.6 wt %, 7.7 wt %, 7.8 wt %, 7.9 wt %, 8 wt %, 8.1 wt %, 8.2 wt %, 8.3 wt %, 8.4 wt %, 8.5 wt %, 8.6 wt %, 8.7 wt %, 8.8 wt %, 8.9 wt %, 9 wt %, 9.1 wt %, 9.2 wt %, 9.3 wt %, 9.4 wt %, 9.5 wt %, 9.6 wt %, 9.7 wt %, 9.8 wt %, 9.9 wt %, or 10 wt %.

Suitable NK-1 receptor antagonists for use in the presently described pharmaceutical compositions, alone or in combination with one or more 5-HT3 receptor antagonists include RP 67580 ((3aR,7aR)-Octahydro-2-[1-imino-2-(2-methoxyphenyl)ethyl]-7,7-diphenyl-4H-isoindol)), WIN 51078 (17-β-Hydroxy-17-α-ethynyl-5-α-androstano[3,2-b]pyrimido[1,2-a]benzimidazole), I-733,060, (((2S,3S)-3-[[3,5-bis(Trifluoromethyl) phenyl]methoxy]-2-phenylpiperidine hydrochloride), I-703,606 (cis-2-(Diphenylmethel)-N-([2-iodophenyl]methyl)-1-azabicyclo(2.2.2)octan-3-amine) MDL 105,212 (R)-1-[2-[3-(3,4-dichlorophenyl)-1-(3,4,5-trimethoxybenzoyl)-pyrrolidin-3-yl]-ethyl]-4-phenylpiperidine-4-carboxamide hydrochloride), serlopitant, maropitant, Antagonist D, aprepitant, fosaprepitant, R116301, CGP49823, CP-96345, CP-99994, GR-203040, MDL-103392, 1-760735, SDZ-NKT-343, nolpitanitium (SR-140333), AV608, rolapitant, SCH 900978, AV608, GSK424887 (GlaxoSmithKline), GSK206136 (GlaxoSmithKline), GR-205171, CP-99994, TAK 637 ((S)-7-(3,5-Bis-trifluoromethyl-benzyl)-9-methyl-5-p-tolyl-8,9,10,11-tetrahydro-7H-1,7,11a-triaza-cycloocta[b]naphthalene-6,12-dione), LY303870 ([(R)-1-[N-(2-methoxybenzyl)acetylamino]-3-(1H-indol-3-yl)-2-[N-(2-(4-(piperidin-1-yl)piperidin-1-yl)acetyl)amino]propane]), LY686017 ((2-chloro-phenyl)-{2-[5-pyridin-4-yl-1-(3,5-bistrifluoromethyl-benzyl)-1H-[1,2,3]triazol-4-yl]-pyridin-3-yl}-methanone), E-6006, casopitant/GW679769 ((2R,4S)-4-(4-acetylpiperazin-1-yl)-N-[(1R)-1-[3,5-bis(trifluoromethyl)phenyl)ethyl]-2-(4-fluoro-2-methylphenyl)-N-methylpiperidine-1-carboxamide), vestipitant, orvepitant and orvepitant maleate, netupitant, ezlopitant, CP-122721, MPC-4505 (Myriad Genetics, Inc.), CP-122721 (Pfizer, Inc.), CJ-1 2,255 (Pfizer, Inc.), SRR 240600 (Sanofi-Aventis), or TA-5538 (Tanabe Seiyaku Co.) including all pharmaceutically acceptable salts thereof.

In embodiments comprising a semi-solid injectable dosage form described herein, the dosage form comprises a NK-1 receptor antagonist incorporated into a polyorthoester delivery vehicle such as those described above. The concentration of the NK-1 receptor antagonist in the composition may vary from about 0.5 wt % to 30 wt %, 0.5 wt % to 20 wt %, 0.5 wt % to 10 wt %, 5 wt % to 20 wt %, 10 wt % to 30 wt %, 2 wt % to 10 wt %, 2 wt % to 5 wt %, or 5 wt % to 15 wt %, and may be 0.5 wt %, 0.75 wt %, 1 wt %, 1.25 wt %, 1.5 wt %, 1.75 wt %, 2 wt %, 2.25 wt %, 2.5 wt %, 2.75 wt %, 3 wt %, 3.25 wt %, 3.5 wt %, 3.75 wt %, 4 wt %, 4.25 wt %, 4.5 wt %, 4.75 wt %, 5 wt %, 5.25 wt %, 5.5 wt %, 5.75 wt %, 6 wt %, 6.25 wt %, 6.5 wt %, 6.75 wt %, 7 wt %, 7.25 wt %, 7.5 wt %, 7.75 wt %, 8 wt %, 8.25 wt %, 8.5 wt %, 8.75 wt %, 9 wt %, 9.25 wt %, 9.5 wt %, 9.75 wt %, 10 wt %, 10.25 wt %, 10.5 wt %, 10.75 wt %, 11 wt %, 11.25 wt %, 11.5 wt %, 11.75 wt %, 12 wt %, 12.25 wt %, 12.5 wt %, 12.75 wt %, 13 wt %, 13.25 wt %, 13.5 wt %, 13.75 wt %, 14 wt %, 14.25 wt %, 14.5 wt %, 14.75 wt %, 15 wt %, 15.25 wt %, 15.5 wt %, 15.75 wt %, 16 wt %, 16.25 wt %, 16.5 wt %, 16.75 wt %, 17 wt %, 17.25 wt %, 17.5 wt %, 17.75 wt %, 18 wt %, 18.25 wt %, 18.5 wt %, 18.75 wt %, 19 wt %, 19.25 wt %, 19.5 wt %, 19.75 wt %, 20 wt %, 20.25 wt %, 20.5 wt %, 20.75 wt %, 21 wt %, 21.25 wt %, 21.5 wt %, 21.75 wt %, 22 wt %, 22.25 wt %, 22.5 wt %, 22.75 wt %, 23 wt %, 23.25 wt %, 23.5 wt %, 23.75 wt %, 24 wt %, 24.25 wt %, 24.5 wt %, 24.75 wt %, 25 wt %, 25.25 wt %, 25.5 wt %, 25.75 wt %, 26 wt %, 26.25 wt %, 26.5 wt %, 26.75 wt %, 27 wt %, 27.25 wt %, 27.5 wt %, 27.75 wt %, 28 wt %, 28.25 wt %, 28.5 wt %, 28.75 wt %, 29 wt %, 29.25 wt %, 29.5 wt %, 29.75 wt %, or 30 wt %.

In one embodiment of the semi-solid injectable compositions, the 5-HT3 receptor antagonists and the NK-1 receptor antagonist are to be presented in a ratio which is consistent with the manifestation of the desired effect. In particular, the ratio by weight of the 5-HT3 receptor antagonists and the other antiemetic agent can suitably be between 0.001:1 and 1:1, 0.001:1 and 0.5:1 and especially between 0.001:1 and 0.25:1

The 5-HT3 antagonists and NK-1 receptor antagonists described herein are also useful for the treatment or prevention of emesis in conjunction with the use of other antiemetic agents known in the art.

Many therapeutic agents are conventionally used in the form of their acid addition salts, as this provides enhanced solubility in aqueous injection media. The antiemetic agent described herein is generally in the free base form.

The therapeutic agent may also be used independently in the form of one or more salts or mixtures of the agent in its unmodified form and in salt form. Suitable pharmaceutically acceptable salts include acid addition salts which may, for example, be formed by mixing a solution of the compound with a solution of a pharmaceutically acceptable acid such as hydrochloric acid, iodic acid, fumaric acid, maleic acid, succinic acid, acetic acid, citric acid, tartaric acid, carbonic acid, phosphoric acid, sulfuric acid and the like. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present disclosure also contemplates salts thereof, preferably non-toxic pharmaceutically acceptable salts thereof, such as the sodium, potassium and calcium salts thereof.

The present disclosure is further directed to a method for ameliorating the symptoms attendant to emesis in a patient comprising administering to the patient a 5-HT3 antagonist and an NK-1 receptor antagonist. In accordance with the present disclosure the 5-HT3 antagonist and the NK-1 receptor antagonist are administered to a patient in a quantity sufficient to treat or prevent the symptoms, and/or underlying etiology associated with emesis in the patient.

Studies to demonstrate the plasma levels of granisetron and aprepitant after subcutaneous administration of a semi-solid formulation which contained both fosaprepitant and granisetron showed that a Tmax for granisetron was approximately 18 hours earlier than the Tmax for aprepitant. It is envisioned that subcutaneous formulations generated according the methods and compositions described herein can provide a Tmax for granisetron at about 0.05 h to 1 h, 0.5 h to 2 h, 0.5 h to 12 h, or 0.5 h to 24 h after subcutaneous administration. A Tmax for aprepitant for the same formulation, when administered subcutaneously, was about 1 h to 36 h, 6 h to 25 h, 12 h to 25 h or 22 h to 26 h after administration. A therapeutically effective Cmax for granisetron provided by formulations described herein may range from about 0.5 ng/mL to 100 ng/mL. A therapeutically effective Cmax achieved for the formulation further comprising fosaprepitant or aprepitant can provide a Cmax of aprepitant ranging from about 500 ng/mL to 3500 ng/mL.

The present disclosure will now be described in connection with certain embodiments, which are not intended to be limiting in scope. On the contrary, the present application covers all alternatives, modifications, and equivalents as included within the scope of the claims. Thus, the following will illustrate the practice of the present disclosure, for the purposes of illustration of certain embodiments and is presented to provide what is believed to be a useful and readily understood description of its procedures and conceptual aspects.

### EXAMPLES

### Example 1

### Preparation of a Semisolid Formulation Containing Aprepitant and Granisetron

375 mg of aprepitant was dissolved in 1.3 g of a polyorthoester-compatible liquid excipient, dimethyl sulfoxide, and heated until completely dissolved. 100 mg of granisetron was added to the mixture and heated until the granisetron and the aprepitant were completely dissolved. The polyorthoester (having a molar ratio of DETOSU:TEG:TEG-diGL of about 90:80:20) was brought to approximately 80° C. and mixed into the solution comprising the granisetron and aprepitant until completely homogenous to provide a semi-solid formulation comprising the illustrative 5-HT3 receptor antagonist and NK-1 receptor antagonist, granisetron and aprepitant, respectively.

### Example 2: In-Vitro Release of Formulation of Aprepitant and Granisetron

A formulation as described in Example 1 was weighed into vials and filled with a phosphate buffered saline solution and 2% CTAB (cetrimonium bromide). The vials were then stored at 37° C. Aliquots were taken daily and analyzed by HPLC to monitor for the release of granisetron and aprepitant from the formulation. See FIG. 3. As shown in FIG. 3, in vitro release of each of the active agents proceeded in a linear fashion up to about 48 hours, with about 65% of each active agent released over this time period. After about 120 hours, about 90% of the aprepipant was released from the formulation, while about 80% of granisetron had been released over the same time frame. Essentially all of the aprepitant is released from the formulation at about 144 hours, while about 90% of the granisetron is released over the same duration.

This data demonstrates that both active agents are released in a sustained fashion over time from the exemplary semi-solid formulation.

### Example 3

### Preparation of a Semi-Solid Formulation Containing Aprepitant

750 mg of aprepitant was dissolved in 2.3 g of a polyorthoester-compatible liquid excipient, dimethyl sulfoxide, and heated until completely dissolved. The polyorthoester described in Example 1 (80:20 TEG-TEG diglycolide diol molar ratio) was brought to approximately 80° C. and mixed into the solution comprised of aprepitant until completely homogenous.

### Example 4

### In Vitro Release of a Formulation Containing Aprepitant

A formulation as described in Example 3 was weighed into vials and filled with a phosphate buffered saline solution and 2% CTAB. The vials were then stored at 37° C. and analyzed by HPLC to monitor for the release of aprepitant from the formulation. See FIG. 4. As shown in FIG. 4, in vitro release of aprepitant proceeded in a linear fashion up to about 72 hours.

### Example 5

### Preparation of a Semisolid Formulation Containing Fosaprepitant

A formulation containing 150 mg of fosaprepitant (a pro-drug form of aprepitant) dissolved in 250 mg of a polyorthoester-compatible liquid excipient, dimethyl sulfoxide, was weighed into an appropriately sized container. The mixture was heated until completely dissolved. The polyorthoester described in Example 1 (80:20 TEG-TEG diglycolide diol ratio) was brought to approximately 80° C. and mixed into the solution of fosaprepitant until completely homogenous.

### Example 6

### Formulation Comprising Fosaprepitant and Granisetron

500 mg of a polyorthoester-compatible liquid excipient, dimethyl sulfoxide, was weighed into an appropriately sized container containing 40 mg of granisetron. The mixture was heated until dissolved and then 300 mg of fosaprepitant (a pro-drug form of aprepitant) was added and completely dissolved to provide a solution comprising granisetron and fosaprepitant. The polyorthoester described in Example 1 (80:20 TEG-TEG diglycolide diol ratio) was brought to approximately 80° C. and mixed into the solution comprising the granisetron and fosaprepitant until completely homogenous.

### Example 7

### In-Vitro Release of Formulation of Fosaprepitant and Granisetron

A formulation as described in Example 6 was weighed into vials and filled with a phosphate buffered saline solution. The vials were then stored at 37° C. and analyzed by HPLC to monitor for the release of granisetron and fosaprepitant from the formulation. See FIG. 1. As shown in FIG. 1, in vitro release of each of the active agents proceeded in a linear fashion up to about 45 hours, with about 25% of each active agent released over this time period. After about 60 hours, nearly 60% of the fosaprepitant (actually, the aprepipant) was released from the formulation, while about 80% of granisetron had been released over the same time frame. Nearly all of the granisetron is released from the formulation at about 100 hours, while about 65% of the aprepitant is released over the same duration.

This data demonstrates that both active agents are released in a sustained fashion over time from the exemplary semi-solid formulation.

### Example 8: Pharmacokinetic Study in Dogs

A subcutaneous injection consisting of 0.5 gm of the formulation described in Example 5 was administered to five dogs. Each injection contained 20 mg/gm of granisetron and 150 mg/gm fosaprepitant. The syringes were stored at 2-8 C and brought to room temperature 1 hour before administration. Each dog received a single subcutaneous injection of the entire contents of 1 syringe (0.5 gm) over the dorsal lumbar muscle on Day 1. Blood was drawn from all dogs at the following time points: T=0, (immediately prior to drug administration), 1, 6, 12, 24, 48, 72, 96, 120, 144, and 168 hours post administration. The plasma samples were analyzed by HPLC for aprepitant and granisetron

As can be seen from the graph, the semi-solid formulation is effective to provide sustained release of both granisetron and aprepitant In looking at FIG. 2, it can be seen Cmax for granisetron is reached at about 6 hours, while Cmax for aprepitant is reached at about 24 hours. The areas under both curves indicate good bioavailability for both drugs upon administration from a semi-solid dosage form such as described herein.

All patent applications, patents, publications, and other published documents mentioned or referred to in this specification are incorporated herein by reference in their entireties, to the same extent as if each individual patent application, patent, publication, and other published document was specifically and individually indicated to be incorporated by reference.

What is claimed is:

1. A pharmaceutical composition comprising a 5-HT3 receptor antagonist or a pharmaceutically acceptable salt thereof, a NK-1 receptor antagonist or a pharmaceutically acceptable salt thereof and a polyorthoester.

2. The pharmaceutical composition of claim 1, comprising about 10-50 weight percent of a polyorthoester-compatible liquid excipient and about 1-5 weight percent granisetron, the polyorthoester comprising subunits selected from: 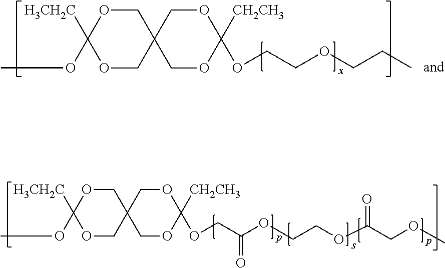
where
x is an integer selected from 1, 2, 3, and 4,
the total amount of p is an integer selected from 1, 2, 3, 4, 5, 6, 7, 8, 9, 10, 11, 12, 13, 14, 15, 16, 17, 18, 19 and 20,
s is an integer selected from 1, 2, 3, and 4,
the mole percentage of α-hydroxyacid containing subunits in the polyorthoester is from about 0.1 to about 25 mole percent,
and the polyorthoester has a molecular weight in a range of about 1000 to 10,000.

3. The composition of claim 1, wherein the 5-HT3 receptor antagonist is granisetron or ondansetron.

4. The composition of claim 1, wherein the NK-1 receptor antagonist is aprepitant or fosaprepitant.

5. The composition of claim 1, wherein the 5-HT3 receptor antagonist is granisetron and the NK-1 receptor antagonist is aprepitant or fosaprepitant.

6. The composition of claim 1, wherein the 5-HT3 receptor antagonist is ondansetron and the NK-1 receptor antagonist is aprepitant or fosaprepitant.

7. The composition of claim 1, wherein the pharmaceutical composition is formulated for subcutaneous administration.

8. A method of treating a subject suffering from chemotherapy-induced nausea and vomiting comprising subcutaneously administering to the subject a pharmaceutical composition comprising a 5-HT3 receptor antagonist or a pharmaceutically acceptable salt thereof, a NK-1 receptor antagonist or a pharmaceutically acceptable salt thereof and a polyorthoester.

9. The method of claim 8, wherein the 5-HT3 receptor antagonist is granisetron or ondansetron and the NK-1 receptor antagonist is aprepitant or fosaprepitant.

10. The method of claim 8, wherein the composition comprises about 10-50 weight percent of a polyorthoester-compatible liquid excipient and about 1-5 weight percent of the 5-HT3 receptor antagonist or a pharmaceutically acceptable salt thereof, the polyorthoester comprising subunits selected from: 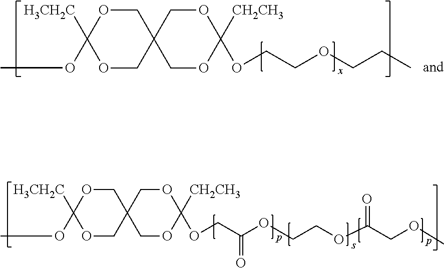
where
x is an integer selected from 1, 2, 3, and 4,
the total amount of p is an integer selected from 1, 2, 3, 4, 5, 6, 7, 8, 9, 10, 11, 12, 13, 14, 15, 16, 17, 18, 19 and 20,
s is an integer selected from 1, 2, 3, and 4,
the mole percentage of α-hydroxyacid containing subunits in the polyorthoester is from about 0.1 to about 25 mole percent,
and the polyorthoester has a molecular weight in a range of about 1000 to 10,000.

11. A method of treating a subject suffering from chemotherapy-induced nausea and vomiting comprising, a first pharmaceutical composition comprising a 5-HT3 receptor antagonist or a pharmaceutically acceptable salt thereof and a polyorthoester, and a second pharmaceutical composition comprising a NK-1 receptor antagonist or a pharmaceutically acceptable salt thereof and a polyorthoester.

12. A subcutaneous dosage form comprising aprepitant or a pharmaceutically acceptable salt thereof and a polyorthoester, wherein the polyorthester comprises subunits selected from: 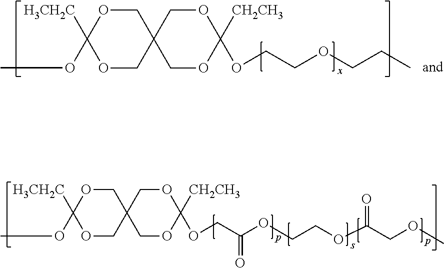
where
x is an integer selected from 1, 2, 3, and 4,
the total amount of p is an integer selected from 1, 2, 3, 4, 5, 6, 7, 8, 9, 10, 11, 12, 13, 14, 15, 16, 17, 18, 19 and 20,
s is an integer selected from 1, 2, 3, and 4,
the mole percentage of α-hydroxyacid containing subunits in the polyorthoester is from about 0.1 to about 25 mole percent,
and wherein the polyorthoester has a molecular weight in a range of about 1000 to 10,000.

13. A subcutaneous dosage form comprising fosaprepitant or a pharmaceutically acceptable salt thereof and a polyorthoester, wherein the polyorthester comprises subunits selected from: 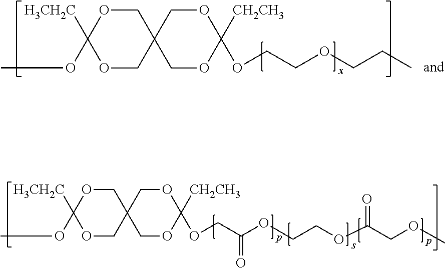
where
x is an integer selected from 1, 2, 3, and 4,
the total amount of p is an integer selected from 1, 2, 3, 4, 5, 6, 7, 8, 9, 10, 11, 12, 13, 14, 15, 16, 17, 18, 19 and 20,
s is an integer selected from 1, 2, 3, and 4,
the mole percentage of α-hydroxyacid containing subunits in the polyorthoester is from about 0.1 to about 25 mole percent,
and wherein the polyorthoester has a molecular weight in a range of about 1000 to 10,000.
